(19) United States
(12) Patent Application Publication
(10) Pub. No.: US 2008/0234508 A1
(43) Pub. Date: Sep. 25, 2008

(54) Process for the Preparation of N(5)-Ethylglutamine

(75) Inventors: Ho Seong Lee, Daejeon (KR); Jeong Ho Song, Daejeon (KR); Ho Cheol Kim, Daejeon (KR)

Correspondence Address: LUCAS & MERCANTI, LLP, 475 PARK AVENUE SOUTH, 15TH FLOOR, NEW YORK, NY, 10016, US

(73) Assignees: DONGBU FINE CHEMICALS CO., LTD, Seoul (KR); CHIROCHEM CO., LTD, Daejeon (KR)

(21) Appl. No.: 11/914,174

(22) PCT Filed: May 18, 2006
(86) PCT No.: PCT/KR06/01870

§ 371 Date: Nov. 12, 2007

(30) Foreign Application Priority Data
May 19, 2005 (KR) 10-2005-0041912

(51) Int. Cl. C07C 237/06; C07C 235/84

(52) U.S. Cl. 560/37; 562/561

(57) ABSTRACT

Disclosed relates to a process for preparing N(5)-ethylglutamines economically without a specific purification process via a simplified and safe process, in which glutamic acid derivatives, represented by formula 1, protected by phthaloyl groups react with ethylamine to cause an amidation and a deprotection reaction in turn under the same reaction condition, thus preparing N(5)-ethylglutamines.

### TECHNICAL FIELD

The present invention relates to a novel process for preparing N(5)-ethylglutamines known as theanine.

### BACKGROUND ART

Theanine is the main component that determines the taste of green tea. It has been known that theanine does numerous functions of physiological activity including: stabilizing the nervous system to reduce stress and enhancing learning ability; inhibiting sleep deprivation action due to caffeine; strengthening the body's natural immune function; preventing dementia; inhibiting apoptosis due to brain infarct; improving premenstrual syndromes; increasing efficacy of anticancer agents; reducing side effects of anticancer agents; and lowering cholesterols. Accordingly, theanine may be used variously as food additives or pharmaceutical materials.

However, since theanine is contained about 0.5 to 2% in dried tea leaves, it is uneconomical to extract theanine from expensive green teas in order to meet the increased demands according to various uses. Accordingly, the necessities of developing chemical syntheses for mass production have been raised.

Synthetic methods known in the past include the method using N-benzyloxycarbonyl-L-glutamic anhydride disclosed in Japanese Patent Publication No. 2001-278848 and the method using N-benzyloxycarbonyl-L-pyrrolidonecarboxylic acid disclosed in Japanese Patent Publication No. 1999-116542, which all have some drawbacks in that expensive catalysts and inflammable hydrogen are used in the process of separating N-protecting groups. Further, the method using L-glutamic acid derivatives protected by t-butoxycarbonyl groups disclosed in Japanese Patent Publication No. 2000-26383 and the method using L-glutamic acid derivatives protected by trityl groups disclosed in Japanese Patent Publication No. 1993-70419 have also some drawbacks in that, since the protecting groups are separated under the acidic condition, the purification process using ion exchange resin should be added thereto and the protecting groups used are expensive. Moreover, the method using L-glutamic acid protected by 2-nitrophenylsulfenyl group disclosed in Japanese Patent Publication No. 2004-203822 includes a simplified purification process; however, it also uses expensive protecting groups. In Chinese Patent Application No. 1560025, the method for preparing theanines has been disclosed comprising: preparing N-phthaloyl-L-glutamic acids, N-phthaloyl-L-glutamic anhydrides in turn from excessive amount of L-glutamic acid; preparing N(5)-ethyl-N′-phthaloyl-L-glutamines using ethylamine solution; and removing the protecting groups using hydrazine solution, thus obtaining theanines. However, such method has numerous drawbacks as follows: firstly, the reaction temperature in preparing N-phthaloyl-L-glutamic acids is very high and it requires an excessive amount of L-glutamic acids; secondly, when the N-phthaloyl-L-glutamic anhydrides react with ethylamine solution, desired N(5)-ethyl-N′-phthaloyl-L-glutamines are prepared along with N-phthaloyl-L-glutamic acids that need a difficult purification process, which can be readily recognized by those having ordinary chemical knowledge; and thirdly, the method of separating the protecting groups using hydrazine solution requires a long reaction time and it is common knowledge that it is prohibited to apply the hydrazine, known as a cancerogenic substance, to the last steps in methods for preparing materials to be administrated to human being in industrial manufactures. Accordingly, an improved method using N-phthaloyl-L-glutamic acid anhydride has been reported by Haining Gu, et al. (Organic Preparations and Procedures International, 182-185, 2004). However, since such method should prepare N(5)-ethyl-N′-phthaloyl-L-glutamine, intermediate, under anhydrous condition, it requires anhydrous acetic anhydride, tetrahydrofuran and gaseous ethylamine, which is an industrially infeasible method. Moreover, all methods described above have a common drawback that requires more than three steps in the reaction process. The method using immobilized enzymes disclosed in Korean Patent Publication No. 2005-0026531 is the most excellent method in numerous preparation methods disclosed so far. However, since such method uses L-glutamine as starting material and glutaminase as immobilized enzyme, it cannot prevent the production of glutamic acid. Accordingly, it is necessary to use ion exchange resins in the purification process and to distill off water, which requires additional processes, thus resulting in the increase of processing cost.

Accordingly, the necessities of developing a simplified and economical process for preparing theanine have been raised.

Conducting researches aimed at overcoming such drawbacks of the well-known methods for preparing theanines, the inventors of the present invention have found a process for preparing theanines economically without a specific purification process via a simplified and safe reaction process using L-glutamic acid derivatives protected by phthaloyl groups, and completed the present invention.

### DISCLOSURE

### Technical Problem

An object of the present invention is to provide a novel process for preparing theanine.

### Technical Solution

To accomplish the object of the present invention, there is provided a process for preparing theanines, in which L-glutamic acid derivatives, represented by formula 1 below, protected by phthaloyl groups react with ethylamine to cause an amidation reaction and a deprotection reaction in turn under the same reaction condition and, subsequently, an appropriate organic solvent is added to the resulting solution to precipitate theanines represented by formula 2 below in a reactor and the precipitated theanines are filtrated, thus preparing theanines economically without a specific purification process via a simplified and safe reaction process.

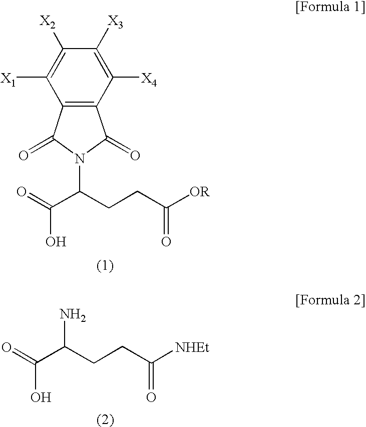

### Advantageous Effects

According to the preparation process of the present invention, it is possible to cause the amidation and the deprotection reaction at the same time by inducing the amidation in an intermediate state, where the phthaloyl groups are not separated completely, under the same condition as the deprotection reaction. Accordingly, the process of the present invention is more simplified and safer compared with the related arts and can be effectively applied to the preparation of theanine economically without a specific purification process.

### BEST MODE

The present invention provides a process for preparing theanines, in which L-glutamic acid derivatives, represented by formula 1 below, protected by phthaloyl groups react with ethylamine to cause an amidation and a deprotection reaction in turn under the same reaction condition and, subsequently, an appropriate organic solvent is added to the reactant solution to precipitate theanines represented by formula 2 below in a reactor and the precipitated theanines are filtrated, thus preparing theanines economically without a specific purification process via a simplified and safe reaction process.

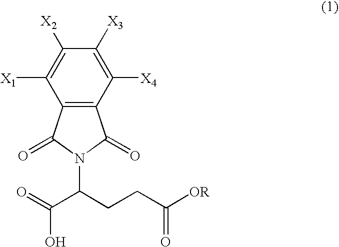

wherein R denotes an alkyl group of C₁˜C₅ or a benzyl group and, preferably, a methyl group or an ethyl group; and X₁, X₂, X₃ and X₄ are one of a hydrogen atom, a halogen atom and a nitro group, independently from one another, and preferably, a hydrogen atom.

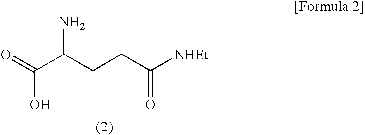

In general, the amino compounds protected by phthaloyl groups are separated using alkylamines (References; Synthesis, 384-387, 1989; Tetrahedron Letters, 4013-4016, 1979). Here, the process of separating the protecting groups is divided into two steps in view of chemical reactions.

First, after the first imide bond is separated under a mild condition, prolonging the reaction time or increasing the temperature makes the second imide bond to be separated. Paying attention to the fact that it requires two chemical steps for separating the phthaloyl groups, the L-glutamic acid derivatives, represented by formula 1, protected by the phthaloyl groups is subjected to a reaction with ethylamine to cause the amidation and the deprotection reaction in turn under the same condition. That is, desired reactions can occur in one reactor using the difference of reaction rates, which will be described more concretely with reference to scheme 1 below.

First, the L-glutamic acid derivatives, represented by formula 1 below, protected by the phthaloyl groups react with a first ethylamine to produce compounds expressed by formula 3 below.

Next, the compounds of formula 3 react with a second ethylamine to produce compounds expressed by formula 4 below. Since such reactions occur almost simultaneously, it is easier to detect the compounds of formula 4 than those of formula 3. The compounds of formula 4 produced like that react with a third ethylamine to be converted all into theanines if the reaction time is prolonged or the reaction temperature is increased. That is, it is possible to reduce the reaction process by causing the amidation simultaneously with the deprotection reaction under the same condition and in an intermediate state, where the protecting groups required to proceeding with the amidation are not separated completely, and lastly by inducing a complete deprotection reaction, thus causing the amidation and the deprotection reaction simultaneously in one rector.

Here, if the reaction of producing compounds expressed by formula 5 below from those of formula 3 below by reacting with the second ethylamine occurs more quickly than that of producing the compounds of formula 4 below from those of formula 3 below, L-pyrrolidonecarboxylic acid expressed by formula 6 below are produced under such reaction conditions, thus not obtaining desired compounds. Thus, only under appropriate reaction conditions, where the reaction rates are remarkably distinguished from each other, as depicted in scheme 1, desired theanines may be obtained.

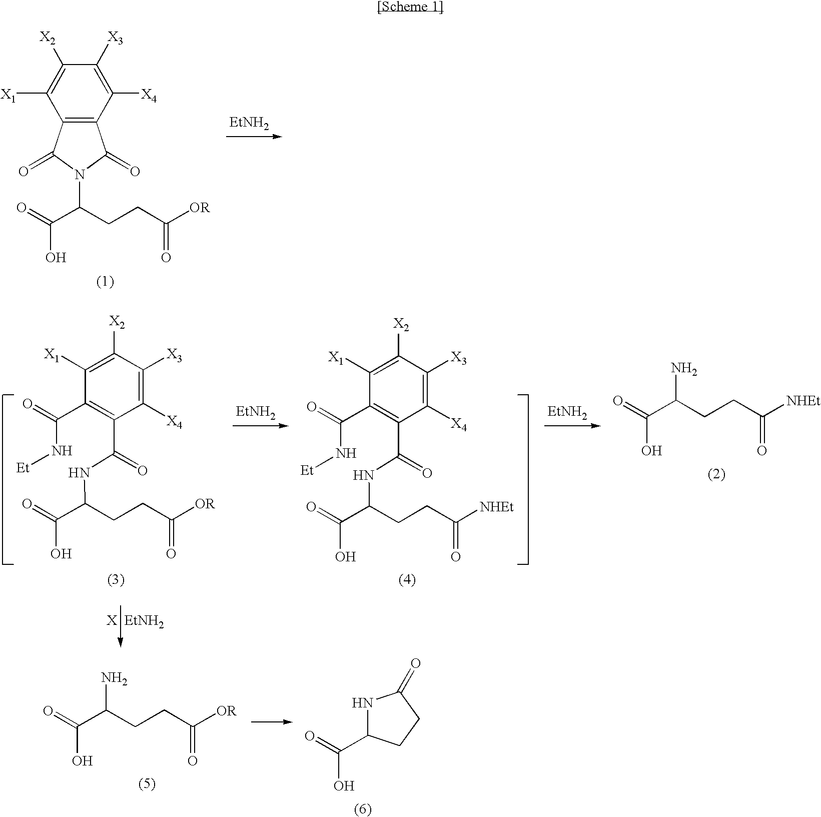

wherein R, X₁, X₂, X₃ and X₄ are the same as defined in formula 1.

Moreover, the phthaloyl derivative used as a protecting group is cheaper than the protecting groups used in the related art and the separated protecting groups can be readily dissolved in general solvents and removed, thus preparing theanines economically without a specific purification process using ion exchange resins.

Hereinafter, the present invention will now be described in detail.

According to the preparation process of the present invention, it is possible to obtain theanines represented by formula 2 via a so-called one pot reaction by reacting the compounds represented by formula 1 with ethylamine of 3 to 30 molar ratio at −20 to 100° C.

Here, 100% anhydrous ethylamine may be subjected to the reaction in the solvent; however, it is desirable to use 30% to 70% ethylamine solution, since the anhydrous ethylamine that is present in a gaseous state at room temperature is hard to handle.

Moreover, the solvent used in accordance with the present invention may include water, methanol, ethanol, isopropanol, butanol, tetrahydrofuran, 1,4-dioxane, dimethylsulfoxide, dimethylformamide, dimethylacetamide, etc. Here, it is possible to use such solvents individually or to mix more than two solvents for such use. Further, the 30 to 70% ethylamine solution used as reactant may be used as a raw material for reaction and a solvent.

After terminating the reaction, theanines may be solidified by adding an appropriate organic solvent to the resulting solution in which the ethylamine existing excessively is removed or not. Here, the organic solvent used may include acetone, methyl ethyl ketone, t-butyl methyl ketone, methanol, ethanol, isopropanol, tetrahydrofuran, 1,4-dioxane, ethyl acetate, methylene chloride, ethylene chloride, etc. Here, it is possible to use such organic solvents individually or to mix more than two solvents for such use.

The compound of formula 1 used in the present invention may be prepared by causing a protection reaction between the compound represented by formula 5, a well-known compound, and a phthaloyl derivative, as depicted in scheme 2 below, applying a method reported by Ajay K. Bose, et al. (Journal of Organic Chemistry, 1335-1388, 1958).

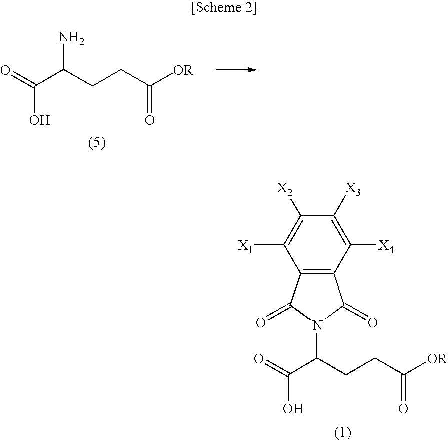

wherein R, X₁, X₂, X₃ and X₄ are the same as defined in formula 1.

The preparation process in accordance with the present invention is carried out as simple as described above, thus providing sufficiently advanced effects that can prepare theanines inexpensively in high yield.

### MODE FOR INVENTION

Hereinafter, the present invention will now be described more fully with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

### PREPARATION EXAMPLE 1

### Preparation of N-phthaloyl-L-glutamic acid 5-methyl ester (formula 1: R=Me, X₁=X₂=X₃=X₄=H)

160 g of toluene was added to 8.1 g of L-glutamic acid 5-methyl ester and 7.4 g of phthalic anhydride. Then, 2.5 g of triethylamine was added thereto and the mixture was stirred at reflux temperature for 6 hours. Here, generated water was removed using a water separator. Toluene was distilled off under reduced pressure and 100 ml of ethyl acetate and 50 ml of 1N hydrochloric acid solution were added thereto. After separating the resulting solution layers, the organic layer was washed with water and dried with magnesium sulfate. The solvent was distilled off under reduced pressure, thus obtaining a target compound in a white solid phase quantitatively.

NMR (CDCl₃)δ(ppm) 9.68 (broad, 1H), 7.90˜7.72 (m, 4H), 5.00 (dd, 1H), 3.62 (s, 3H), 2.69˜2.44 (m, 2H), 2.41 (m, 2H)

### PREPARATION EXAMPLE 2

### Preparation of N-Phthaloyl-L-glutamic acid 5-ethyl ester (formula 1: R=Et, X₁=X₂=X₃=X₄=H)

Using 8.8 g of L-glutamic acid 5-ethyl ester instead of L-glutamic acid 5-methyl ester, a target compound in a white oil phase was obtained quantitatively via the same process as preparation example 1.

NMR (CDCl₃)δ(ppm): 10.62 (broad, 1H), 7.90˜7.72 (m, 4H), 5.00 (dd, 1H), 4.04 (q, 2H), 2.68-2.40 (m, 2H), 2.41 (m, 2H), 1.20 (t, 3H)

### PREPARATION EXAMPLE 3

### Preparation of N-tetrachlorophthaloyl-L-glutamic acid 5-methyl ester (formula 1: R=Me, X₁=X₂=X₃=X₄=Cl)

Using 14.3 g of tetrachlorophthalic anhydride instead of phthalic anhydride, a target compound was obtained quantitatively in a white solid phase via the same process as preparation example 1.

NMR (DMSO-d₆)δ(ppm): 4.86 (dd, 1H), 3.56 (s, 3H), 2.52˜2.15(m, 4H)

### PREPARATION EXAMPLE 4

### Preparation of N-Phthaloyl-L-glutamic acid 5-benzyl ester (formula 1: R═CH₂Ph, X₁=X₂=X₃=X₄=H)

Using 11.9 g of L-glutamic acid 5-benzyl ester instead of L-glutamic acid 5-methyl ester, a target compound (16.9 g, 92%) was obtained in a white oil phase via the same process as preparation example 1.

NMR (CDCl₃)δ(ppm) 10.82 (broad, 1H), 7.87˜7.69 (m, 4H), 7.31 (m, 5H), 5.03 (s, 2H), 5.00 (dd, 1H), 2.68˜2.40 (m, 2H), 2.41 (m, 2H)

### EXAMPLE 1

### Preparation of N(5)-ethyl-L-glutamine (theanine)

5.8 g of N-phthaloyl-L-glutamic acid 5-methyl ester (formula 1: R=Me, X₁=X₂=X₃=X₄=H) was added to 12.9 g of 70% ethylamine solution at 0° C. and stirred for one hour. Then, the reaction temperature was raised up to 20° C. After stirring the resulting solution at 20° C. for 22 hours, the ethylamine existing excessively was removed under reduced pressure. After adding 38.6 g of acetone to the resulting solution, the pH of the solution was regulated as 5 to 6 using acetic acid. Then, the resulting solution was stirred for one hour. Produced solids were filtrated and washed with ethanol. The filtrated white solids were dried to obtain a target compound (3.1 g, 89%).

NMR (D₂O)δ(ppm): 3.77 (t, 1H), 3.20 (q, 2H), 2.40 (m, 2H), 2.13 (dd, 2H), 1.11 (t, 3H)

[α]²⁰+8.0° (c=5, H₂O)

### EXAMPLE 2

### Preparation of N-(2-ethylcarbamoylbenzoyl)-L-glutamic acid 5-methyl ester (formula 3: R=Me, X₁=X₂=X₃=X₄=H) and N(5)-ethyl-N′-(2-ethylcarbomoylbenzoyl)-L-glutamine (formula 4: X₁=X₂=X₃=X₄=H)

0.6 g of N-phthaloyl-L-glutamic acid 5-methyl ester (formula 1: R=Me, X₁=X₂=X₃=X₄=H) was added to 1.3 g of 70% ethylamine solution at 0 and stirred for one hour. Then, the ethylamine existing excessively was removed under reduced pressure. Silica gel column chromatography was carried out for the resulting solution to collect two materials (Rf=0.29 and 0.20, respectively) using a developing solvent of ethyl acetate:methanol (6:4). Solvent first eluted was removed under reduced pressure to obtain N-(2-ethylcarbamoylbenzoyl)-L-glutamic acid 5-methyl ester in a white solid phase, and solvent later eluted was removed under reduced pressure to obtain N(5)-ethyl-N′-(2-ethylcarbamoylbenzoyl)-L-glutamine in a white solid phase.

N-(2-ethylcarbamoylbenzoyl)-L-glutamic acid 5-methyl ester

NMR (DMSO-d₆)δ(ppm): 8.46 (t, 1H), 7.82 (d, 1H), 7.49 (m, 4H), 4.27 (broad, 1H), 3.58 (s, 3H), 3.20 (m, 2H), 2.40 (m, 2H), 2.10 (m, 1H), 1.89 (m, 1H), 1.08 (t, 3H)

N(5)-ethyl-N′-(2-ethylcarbamoylbenzoyl)-L-glutamine

NMR (DMSO-d₆)δ(ppm): 8.40 (t, 1H), 7.96 (d, 1H), 7.84 (t, 1H), 7.48 (m, 4H), 4.13 (m, 1H), 3.22 (m, 2H), 3.08 (m, 2H), 2.25˜1.95 (m, 3H), 1.82 (m, 1H), 1.08 (t, 3H), 0.99 (t, 3H)

### EXAMPLE 3

### Preparation of N-(2-ethylcarbamoylbenzoyl)-L-glutamic acid 5-methyl ester (formula 3: R=Me, X₁=X₂=X₃=X₄=H)

0.13 g of 70% ethylamine solution and 0.17 g of water were added to 0.29 g of N-phthaloyl-L-glutamic acid 5-methyl ester (formula 1: R=Me, X₁=X₂=X₃=X₄=H) and stirred at 20° C. for 22 hours. Then, the ethylamine existing excessively was removed under reduced pressure. Silica gel column chromatography was carried out for the resulting solution to collect a material of which Rf is 0.29 using a developing solvent of ethyl acetate:methanol (6:4). Subsequently, solvent was removed under reduced pressure to obtain a target compound (0.2 g of white solids).

NMR (DMSO-d₆)δ(ppm): 8.46 (t, 1H), 7.82 (d, 1H), 7.49 (m, 4H), 4.27 (broad, 1H), 3.58 (s, 3H), 3.20 (m, 2H), 2.40 (m, 2H), 2.10 (m, 1H), 1.89 (m, 1H), 1.08 (t, 3H)

### EXAMPLE 4

### Preparation of N(5)-ethyl-D-glutamine (D-isomer of theanine)

6.1 g of N-phthaloyl-D-glutamic acid 5-ethyl ester (formula 1: R=Et, X₁=X₂=X₃=X₄=H) was added to 12.9 g of 70% ethylamine solution at 0° C. and stirred for one hour. Then, the reaction temperature was raised up to 20° C. After stirring the resulting solution at 20° C. for 22 hours, the ethylamine existing excessively was removed under reduced pressure. After adding 30.9 g of ethanol to the resulting solution, the pH of the solution was regulated as 5 to 6 using acetic acid. Subsequently, the resulting solution was refluxed for one hour and cooled to room temperature. Produced solids were filtrated and washed with ethanol. The filtrated white solids were dried to obtain a target compound (2.8 g, 80%).

NMR (D₂O)δ(ppm): 3.77 (t, 1H), 3.20 (q, 2H), 2.40 (m, 2H), 2.13 (dd, 2H), 1.11 (t, 3H)

[α]²⁰ −8.0° (c=5, H₂O)

### EXAMPLE 5

### Preparation of N(5)-ethyl-DL-glutamine (Racemic Mixture of Theanine)

7.3 g of N-phthaloyl-DL-glutamic acid 5-benzyl ester (formula 1: R═CH₂Ph, X₁=X₂=X₃=X₄=H) was added to 12.9 g of 70% ethylamine solution at 0 and stirred for one hour. Then, the reaction temperature was raised up to 20° C. After stirring the resulting solution at 20° C. for 22 hours, the ethylamine existing excessively was removed under reduced pressure. After adding 30.9 g of isopropanol to the resulting solution, the pH of the solution was regulated as 5 to 6 using acetic acid. Subsequently, the resulting solution was refluxed for one hour and cooled to room temperature. Produced solids were filtrated and washed with ethanol. The filtrated white solids were dried to obtain the captioned compound (2.8 g, 80%).

NMR (D₂O)δ(ppm): 3.77 (t, 1H), 3.20 (q, 2H), 2.40 (m, 2H), 2.13 (dd, 2H), 1.11 (t, 3H)

[α]²⁰ 0.0° (c=5, H₂O)

Although the present invention has been described with reference to certain exemplary embodiments thereof, it will be understood by those skilled in the art that a variety of modifications may be made therein without departing from the spirit or scope of the present invention defined by the appended claims and their equivalents.

1. A process for preparing N(5)-ethylglutamine, represented by formula 2 below, by reacting a compound, represented by formula I below, with ethylamine to cause an amidation and a deprotection reaction in turn under the same reaction condition: 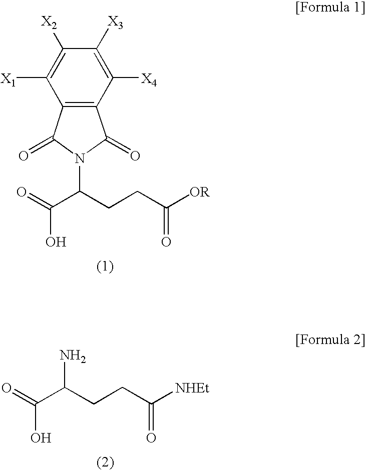
wherein, in formula I, R denotes an alkyl group of C_{1˜C}₅ or a benzyl group; and X₁, X₂, X₃ and X₄ are respectively one selected from the group consisting of a hydrogen atom, a halogen atom and a nitro group.

2. The process for preparing N(5)-ethylglutamine as recited in claim 1, wherein the compound represented by formula 1 or 2 is a racemic mixture or a chiral compound.

3. The process for preparing N(5)-ethylglutamine as recited in claim 1, wherein R is a methyl group or an ethyl group; and X₁, X₂, X₃ and X₄ are hydrogen atoms.

4. A process for preparing N(5)-ethylglutamine by reacting a compound, represented by formula 1, with ethylamine to produce a compound, represented by formula 3, which then reacts with ethylamine to produce a compound, represented by formula 4, as depicted in scheme I below: 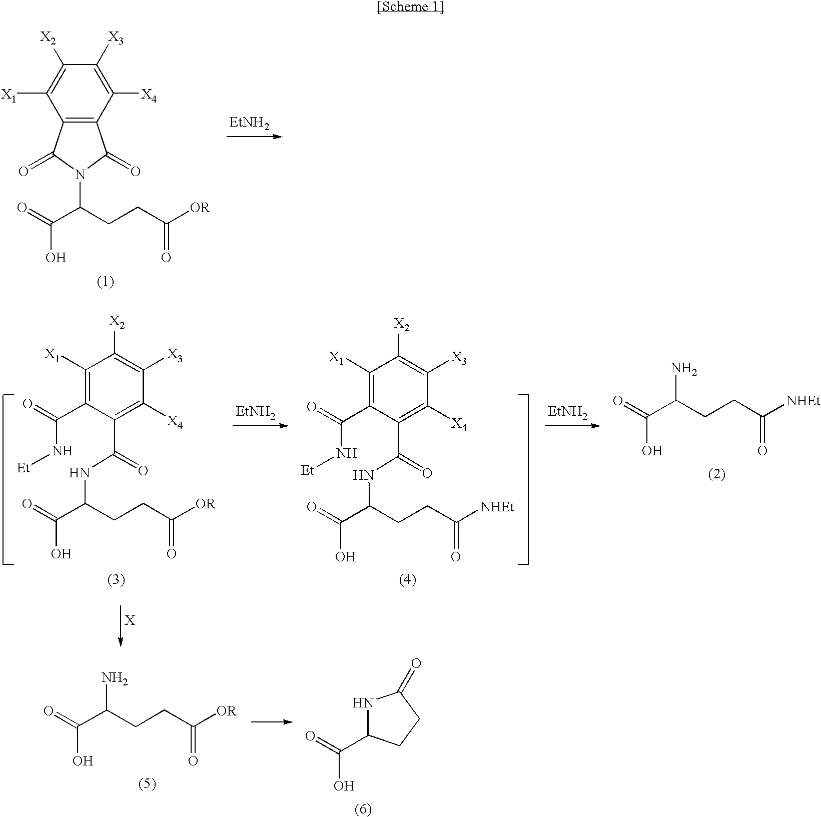
wherein
R is an alkyl group of C₁˜C₅ or a benzyl group, and
X₁, X₂, X₃ and X₄ are independently selected from the group consisting of a hydrogen atom, a halogen atom and a nitro group.

5. The process for preparing N(S)-ethylglutamine as recited in claim 4, wherein the compound, represented by formula 1, 2, 3 or 4, is one of a racemic compound and a chiral compound.

6. The process for preparing N(5)-ethylglutamine as recited in claim 4, wherein R is a methyl group or an ethyl group; and X₁, X₂, X₃ and X₄ are hydrogen atoms.

7. A compound, represented by formula 3 below: 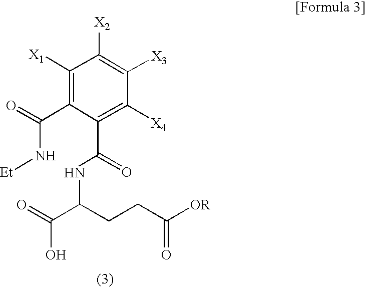
wherein
R is an alkyl group of C₁˜C₅ or a benzyl group; and
X₁, X₂, X₃ and X₄ are independently selected from the group consisting of a hydrogen atom, a halogen atom and a nitro group.

8. A compound, represented by formula 4 below: 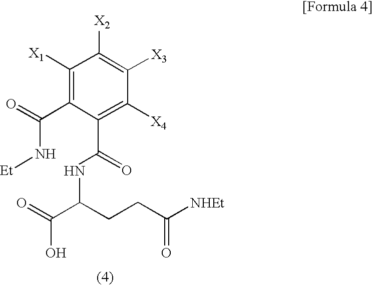
wherein
R is an alkyl group of C₁˜C₅ or a benzyl group; and
X₁, X₂, X₃ and X₄ are independently selected from the group consisting of a hydrogen atom, a halogen atom and a nitro group.

9. The compound as recited in claim 7, wherein the compound, represented by formula 3 or 4, is a racemic mixture or a chiral compound, respectively.

10. The compound as recited in claim 7, wherein R is a methyl group or an ethyl group; and X₁, X₂, X₃ and X₄ are hydrogen atoms.

11. The compound as recited in claim 8, wherein the compound of formula 3 is a racemic mixture or a chiral compound; and the compound of formula 4 is a racemic mixture of a chiral compound.

12. The compound as recited in claim 8, wherein R is a methyl group or an ethyl group; and X₁, X₂, X₃ and X₄ are hydrogen atoms.
